(19) United States
(12) Patent Application Publication
(10) Pub. No.: US 2026/0345124 A1
(43) Pub. Date: Nov. 5, 2026

(54) ULTRASONIC DIAGNOSTIC APPARATUS, MEDICAL IMAGE PROCESSING APPARATUS AND NON-TRANSITORY COMPUTER READABLE STORAGE MEDIUM

(71) Applicant: CANON KABUSHIKI KAISHA, Tokyo (JP)

(72) Inventors: Masaki WATANABE, Tochigi (JP); Yukari SONE, Tochigi (JP)

(73) Assignee: CANON KABUSHIKI KAISHA, Tokyo (JP)

(21) Appl. No.: 19/659,058

(22) Filed: Apr. 27, 2026

(30) Foreign Application Priority Data
Apr. 30, 2025 (JP) 2025-075496

(51) Int. Cl. A61B 8/00

(52) U.S. Cl. CPC A61B 8/469; A61B 8/485; A61B 8/5223

(57) ABSTRACT

An ultrasonic diagnostic apparatus according to an embodiment includes an identification unit, a calculation unit, and a setting unit. The identification unit identifies a target site included in ultrasonic image data of a subject. The calculation unit calculates respective index values for a plurality of positions that are specified with the target site serving as a reference, the index values representing reliability of a tissue characterization parameter represented by the ultrasonic image data. The setting unit sets, based on the calculated results, a region of interest for observing the tissue characterization parameter onto an image represented by the ultrasonic image data.

### CROSS REFERENCE TO RELATED APPLICATIONS

This application is based upon and claims the benefit of priority from the prior Japanese Patent Application No. 2025-075496, filed on Apr. 30, 2025, the entire contents of which are incorporated herein by reference.

### FIELD

Embodiments described in the present specification and drawings relate to an ultrasonic diagnostic apparatus, a medical image processing apparatus and a non-transitory computer readable storage medium.

### BACKGROUND

An application for indexing tissue characterization of a subject using a medical image diagnostic apparatus, such as an ultrasonic diagnostic apparatus, a magnetic resonance imaging (MRI) apparatus, and a computed tomography (CT) apparatus, has been developed. An index value calculated by such an application is presented to a user as an image or numerical information, and is employed for diagnosis or the like.

In order to accurately index the tissue characterization of the subject using this application, that is, in order to accurately analyze the tissue characterization of the subject, it is necessary to set a region of interest (hereinafter, also denoted as an ROI) to a position that avoids a structure, such as a blood vessel, or to a position where there are a few structures. The setting practices of the region of interest are conventionally performed manually by the user and involve the subjectivity of the user, which may affect accuracy of the tissue characterization analysis.

### BRIEF DESCRIPTION OF THE DRAWINGS

FIG. 1 is a block diagram illustrating an example of a configuration of an ultrasonic diagnostic apparatus according to a first embodiment.

FIG. 2 is a flowchart for explaining observation ROI setting processing executed in the ultrasonic diagnostic apparatus according to the first embodiment.

FIG. 3 is a diagram illustrating an example of B-mode image data according to the first embodiment.

FIG. 4 is a diagram illustrating an example of normalized local variance image data according to the first embodiment.

FIG. 5 is a diagram for explaining processing of identifying a target site included in the B-mode image data.

FIG. 6 is a diagram for explaining processing of identifying the target site included in the B-mode image data.

FIG. 7 is a diagram for explaining processing of identifying the target site included in the B-mode image data.

FIG. 8 is a diagram for explaining processing of calculating index values for a plurality of positions that are specified with the target site serving as a reference, in the first embodiment.

FIG. 9 is a diagram illustrating an example of results of calculating the index values for the plurality of respective positions.

FIG. 10 is a diagram illustrating an example of a selection result according to the first embodiment.

FIG. 11 is a diagram illustrating an example of a region of interest set onto an image represented by the B-mode image data in the first embodiment.

FIG. 12 is a diagram illustrating an example of an image represented by tissue characterization image data according to the first embodiment.

FIG. 13 is a diagram illustrating an example of identification information displayed in an observation ROI.

FIG. 14 is a flowchart for explaining measurement ROI setting processing executed in an ultrasonic diagnostic apparatus according to a second embodiment.

FIG. 15 is a flowchart for explaining ROI setting processing executed in an ultrasonic diagnostic apparatus according to a third embodiment.

FIG. 16 is a diagram for explaining processing of calculating index values for a plurality of positions that are specified with the target site serving as the reference in the third embodiment.

FIG. 17 is a diagram illustrating an example of measurement ROIs and an observation ROI that are set onto an image represented by B-mode image data in the third embodiment.

FIG. 18 is a flowchart for explaining measurement ROI setting processing executed in an ultrasonic diagnostic apparatus according to a second modification.

FIG. 19 is a block diagram illustrating an example of a configuration of a medical image processing apparatus according to a fourth embodiment.

FIG. 20 is a diagram illustrating an example of a case where an observation ROI and measurement ROIs are regions for measuring tissue characterization of SWE.

### DETAILED DESCRIPTION

Hereinafter, respective embodiments of the ultrasonic diagnostic apparatus, the medical image processing apparatus and the non-transitory computer readable storage will be described with reference to the accompanying drawings. In the embodiments below, the same reference signs are given for identical components in terms of configuration and function, and duplicate description is omitted.

### First Embodiment

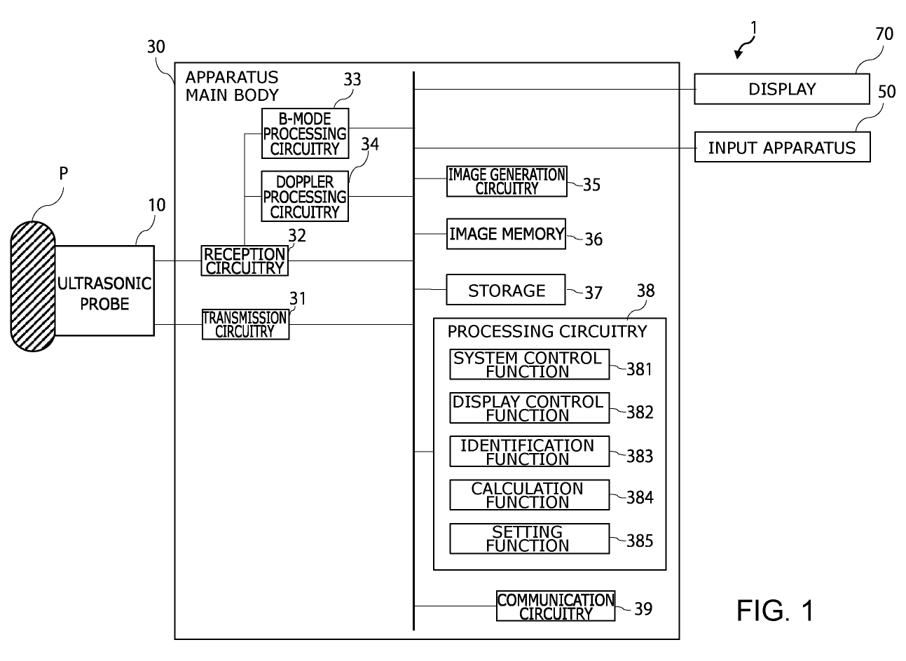

FIG. 1 is a block diagram illustrating an example of a configuration of an ultrasonic diagnostic apparatus according to a first embodiment. As illustrated in FIG. 1, an ultrasonic diagnostic apparatus 1 includes an ultrasonic probe 10, an apparatus main body 30, an input apparatus 50, and a display 70.

The ultrasonic probe 10 includes, for example, a plurality of piezoelectric vibrators. The plurality of piezoelectric vibrators generate ultrasonic waves based on a drive signal supplied from transmission circuitry 31 included in the apparatus main body 30 to be described later. The ultrasonic probe 10 receives a reflected wave from a subject P and converts the reflected wave into an electrical signal. The ultrasonic probe 10 includes, for example, matching layers provided on the piezoelectric vibrators, a backing material for preventing propagation of ultrasonic waves rearward from the piezoelectric vibrators, and the like.

When ultrasonic waves are transmitted from the ultrasonic probe 10 to the subject P, the transmitted ultrasonic waves are reflected one after another on a discontinuous surface of an acoustic impedance in a body tissue of the subject P, and are received as reflected wave signals by the plurality of piezoelectric vibrators included in the ultrasonic probe 10. An amplitude of the received reflected wave signals depends on a difference in acoustic impedance at the discontinuous surface at which the ultrasonic waves are reflected. The reflected wave signals in a case where transmitted ultrasonic pulses are reflected by a moving blood flow, a surface of a heart wall, or the like receive a frequency shift depending on a velocity component with respect to a transmission direction of an ultrasonic wave of a moving body, due to the Doppler effect.

The ultrasonic probe 10 is connected to the apparatus main body 30 in a wired or wireless manner, and communicates with the apparatus main body 30. When performing communication in a wired manner, the ultrasonic probe 10 is detachably connected to the apparatus main body 30. When scanning of a two-dimensional region in the subject P (two-dimensional scanning) is performed, a user connects, for example, a 1D array probe in which a plurality of piezoelectric vibrators are arranged in a line, to the apparatus main body 30 as the ultrasonic probe 10. The 1D array probe is a linear-type ultrasonic probe, a convex-type ultrasonic probe, a sector-type ultrasonic probe, or the like. When scanning of a three-dimensional region in the subject P (three-dimensional scanning) is performed, the user connects, for example, a mechanical 4D probe or a 2D array probe to the apparatus main body 30 as the ultrasonic probe 10. The mechanical 4D probe can perform two-dimensional scanning using a plurality of piezoelectric vibrators arranged in a line like the 1D array probe, and can perform three-dimensional scanning by swinging the plurality of piezoelectric vibrators at a predetermined angle (swing angle). The 2D array probe can perform three-dimensional scanning by a plurality of piezoelectric vibrators arranged in a matrix, and can perform two-dimensional scanning by focusing and transmitting ultrasonic waves. The 2D array probe can simultaneously perform two-dimensional scanning of a plurality of cross sections.

The apparatus main body 30 generates ultrasonic image data based on a signal from the ultrasonic probe 10. Specifically, the apparatus main body 30 can generate two-dimensional ultrasonic image data based on a reflected wave signal relevant to a two-dimensional region of the subject P received by the ultrasonic probe 10. The apparatus main body 30 can generate three-dimensional ultrasonic image data based on a reflected wave signal relevant to a three-dimensional region of the subject P received by the ultrasonic probe 10. As illustrated in FIG. 1, the apparatus main body 30 includes the transmission circuitry 31, reception circuitry 32, B-mode processing circuitry 33, Doppler processing circuitry 34, image generation circuitry 35, an image memory 36, storage 37, processing circuitry 38, and communication circuitry 39.

The transmission circuitry 31 includes a pulse generator, a transmission delay unit, a pulser, and the like, and supplies a drive signal to the ultrasonic probe 10. The pulse generator repeatedly generates rate pulses for forming transmission ultrasonic waves at a predetermined rate frequency. The transmission delay unit focuses ultrasonic waves generated from the ultrasonic probe 10 into a beam shape, and gives, to each rate pulse generated by the pulse generator, a delay time for each piezoelectric vibrator necessary to determine transmission directivity. The pulser applies a drive signal (drive pulse) to the ultrasonic probe 10 at a timing based on the rate pulse. That is, the transmission delay unit freely adjusts the transmission direction of the ultrasonic wave transmitted from a piezoelectric vibrator surface by changing the delay time given to each rate pulse.

The transmission circuitry 31 has a function of enabling a transmission frequency, a transmission driving voltage, and the like to be instantaneously changed in order to execute a predetermined scan sequence based on an instruction from the processing circuitry 38 to be described later. In particular, the transmission driving voltage is changed by linear-amplifier-type transmission circuitry capable of instantaneously switching the value thereof or by a mechanism for electrically switching a plurality of power supply units.

The reception circuitry 32 includes a preamplifier, an analog-to-digital (A-D) converter, a reception delay unit, an adder, and the like, and performs various processing on the reflected wave signal received by the ultrasonic probe 10, thereby generating reflected wave data. The preamplifier amplifies the reflected wave signal for each channel and performs gain adjustment (gain correction). The A-D converter A-D converts the gain-corrected reflected wave signal into a digital signal by A-D converting the gain-corrected reflected wave signal. The reception delay unit gives a delay time necessary to determine reception directivity. The adder performs processing of adding reflected wave signals processed by the reception delay unit, and generates reflected wave data. Then, the adder outputs the generated reflected wave data to the B-mode processing circuitry 33 and the Doppler processing circuitry 34.

When the subject P is two-dimensionally scanned, the transmission circuitry 31 causes the ultrasonic probe 10 to transmit a two-dimensional ultrasonic beam. Then, the reception circuitry 32 generates two-dimensional reflected wave data from a two-dimensional reflected wave signal received by the ultrasonic probe 10. When the subject P is three-dimensionally scanned, the transmission circuitry 31 according to the present embodiment causes the ultrasonic probe 10 to transmit a three-dimensional ultrasonic beam. Then, the reception circuitry 32 generates three-dimensional reflected wave data from a three-dimensional reflected wave signal received by the ultrasonic probe 10.

The B-mode processing circuitry 33 receives the reflected wave data from the reception circuitry 32, performs logarithmic amplification, envelope detection processing, and the like, and generates data (B-mode data) in which a signal intensity is expressed by brightness derived from luminance.

The Doppler processing circuitry 34 performs frequency analysis on velocity information from the reflected wave data received from the reception circuitry 32, extracts a blood flow, a tissue, and a contrast medium echo component through the Doppler effect, and generates data (Doppler data) obtained by extracting moving body information, such as velocity, dispersion, and power, for multiple points. Here, the moving body is, for example, a blood flow, a tissue of an organ that periodically moves, such as a heart wall, or a contrast medium.

The B-mode processing circuitry 33 and the Doppler processing circuitry 34 can process both types of the two-dimensional reflected wave data and the three-dimensional reflected wave data. That is, the B-mode processing circuitry 33 generates two-dimensional B-mode data from two-dimensional reflected wave data, and generates three-dimensional B-mode data from three-dimensional reflected wave data. The Doppler processing circuitry 34 generates two-dimensional Doppler data from two-dimensional reflected wave data, and generates three-dimensional Doppler data from three-dimensional reflected wave data.

The image generation circuitry 35 generates ultrasonic image data expressed in a predetermined luminance range based on a signal received by the ultrasonic probe 10. For example, the image generation circuitry 35 generates, as the ultrasonic image data, two-dimensional B-mode image data in which the intensity of the reflected wave is represented by luminance from the two-dimensional B-mode data generated by the B-mode processing circuitry 33. The image generation circuitry 35 generates, as the ultrasonic image data, two-dimensional color Doppler image data serving as average speed image data, variance image data, power image data, or a combined image thereof that represents the moving body information from the two-dimensional Doppler data generated by the Doppler processing circuitry 34.

Here, the image generation circuitry 35 generally converts (scan converts) a scanning line signal sequence in ultrasonic scanning into a scanning line signal sequence in a video format typified by a television or the like, and generates display ultrasonic image data. For example, the image generation circuitry 35 generates the display ultrasonic image data by performing coordinate conversion according to a mode of ultrasonic wave scanning by the ultrasonic probe 10. For example, the image generation circuitry 35 performs, as various types of image processing other than the scan conversion, image processing (smoothing processing) of regenerating an average value image based on luminance by using a plurality of image frames after scan conversion, image processing (edge enhancement processing) using a differential filter in the image, and the like.

That is, the B-mode data and the Doppler data are data before the scan conversion processing, and the data generated by the image generation circuitry 35 is display ultrasonic image data after the scan conversion processing. The B-mode data and the Doppler data are also referred to as raw data. The image generation circuitry 35 generates the two-dimensional B-mode image data or the two-dimensional color Doppler image data, which is two-dimensional ultrasonic image data, from the two-dimensional B-mode data or the two-dimensional Doppler data, which is raw data. The image generation circuitry 35 can also generate, for example, a superimposed image in which a color Doppler image is superimposed on the two-dimensional B-mode image data.

For example, the image generation circuitry 35 generates M-mode image data from time-series data of the B-mode data on one scanning line generated by the B-mode processing circuitry 33. The image generation circuitry 35 generates a Doppler waveform obtained by plotting a blood flow and tissue velocity information in time series from the Doppler data generated by the Doppler processing circuitry 34.

In addition, the image generation circuitry 35 generates, as the ultrasonic image data, tissue characterization image data indicating information on tissue characterization of the subject P. The tissue characterization refers to, for example, properties and states of a tissue. The tissue characterization image data is, for example, elasticity image data related to an image representing elasticity (stiffness) of a tissue, viscosity image data related to an image representing viscosity of a tissue, attenuation image data related to an image representing a degree of attenuation of ultrasonic waves in a tissue to be examined, variance image data related to an image representing a degree of variance of a signal reflected from the inside of a tissue of the subject P, and the like.

The elasticity image data is, for example, image data obtained by indexing the stiffness of a tissue. When a “push pulse” having a high acoustic radiation force is transmitted from the ultrasonic probe 10, a part of the tissue is deformed and a shear wave is generated. The elasticity image data is generated by observing, through transmission and reception of a “tracking pulse”, a state in which the generated shear wave is propagating. More specifically, the elasticity image data is generated, for example, by processing a reception signal or Doppler data generated by transmission and reception of a tracking pulse, calculating a temporal change of displacement for each position in a region to be imaged, and calculating a propagation speed of a shear wave based on the temporal change of displacement. Here, the push pulse is a focused ultrasonic pulse that generates, based on the acoustic radiation force, a lateral wave called a shear wave in a living tissue, and is an example of an ultrasonic wave for generating the shear wave. The tracking pulse is an ultrasonic pulse for observing the shear wave, and is an example of an ultrasonic wave for observing the shear wave. The elasticity image data may be generated, after a dynamic load is applied to a tissue of the subject, based on a change (distortion) in a shape of the tissue to which the load is applied.

The viscosity image data is, for example, image data obtained by indexing viscosity of a tissue. The viscosity image data is generated, for example, based on a frequency and a propagation speed of a shear wave. The viscosity image data is generated by processing a reception signal or Doppler data generated by transmission and reception of a tracking pulse, for example, similarly to the elasticity image data.

The attenuation image data is, for example, image data obtained by indexing the degree of attenuation of an ultrasonic wave in a tissue to be examined. The attenuation image data is generated, for example, by processing the reception signal generated by the reception circuitry 32, the B-mode data generated by the B-mode processing circuitry 33, or the B-mode image data generated by the image generation circuitry 35. More specifically, the attenuation image data is generated, for example, by transmitting and receiving a narrow-band ultrasonic pulse, reducing an influence of gain correction, a sound field, or the like from the acquired reception signal, B-mode data, or B-mode image data, and calculating an amount of change in signal intensity in a depth direction. For example, the attenuation image data is generated by transmitting and receiving a plurality of ultrasonic pulses having different center frequencies, comparing how much the intensities of a plurality of acquired reception signals change in the depth direction, and estimating an amount of attenuation unique to the subject.

The variance image data is, for example, image data obtained by indexing the degree of variance of a signal reflected from the inside of a tissue of the subject P. The variance image data is generated, for example, by processing the reception signal generated by the reception circuitry 32, the B-mode data generated by the B-mode processing circuitry 33, or the B-mode image data generated by the image generation circuitry 35. More specifically, the variance image data is generated, for example, by calculating a degree of deviation, from the Rayleigh distribution, of a signal amplitude distribution of the reception signal generated from the reception circuitry 32 for each local area.

The generated elasticity image data, viscosity image data, attenuation image data, and variance image data are each associated with a pixel value that is mapped to a predetermined color, depending on an index value (tissue characterization parameter) that represents tissue characterization calculated for each pixel. The generated elasticity image data, viscosity image data, attenuation image data, and variance image data are each displayed on the display 70 as color maps.

The tissue characterization parameter is calculated for each of a plurality of positions in a region of interest by analyzing a result of a scan performed on the subject. The tissue characterization parameter is, for example, an index value representing the stiffness of a tissue, an index value representing the viscosity of a tissue, an index value representing the degree of attenuation of an ultrasonic wave in a tissue to be examined, a ratio of a reflection intensity of an ultrasonic wave in the liver and the kidney, the degree of variance of a signal reflected from the inside of a tissue of the subject P, and a strain ratio representing a ratio of stiffness of a tissue at a site of interest to stiffness of a predetermined tissue serving as a reference.

The index value representing the stiffness of a tissue is used, for example, when hepatic fibrosis is diagnosed. The index value representing the stiffness of a tissue is represented by, for example, an elastic modulus. The elastic modulus takes a larger value as hepatic fibrosis progresses, for example. The index value of the elasticity is not limited to the elastic modulus. The index value of the elasticity may be, for example, the propagation speed itself. The index value representing the stiffness of a tissue corresponds to an elasticity parameter in the present embodiment. Hereunder, the index value representing the stiffness of a tissue is also expressed as an elasticity index value.

The index value representing the viscosity of a tissue is used when a case is diagnosed where the viscosity is considered to considerably increase due to necrosis and inflammation, for example, acute hepatitis is diagnosed. The index value representing the viscosity of a tissue is represented by, for example, a viscosity coefficient. The viscosity coefficient takes a larger value, for example, in a case of suffering from acute hepatitis. The index value of the viscosity is not limited to viscosity coefficient. The index value of the viscosity may be, for example, a slope or the like in a distribution of the frequency and the propagation speed. The index value representing the viscosity of a tissue corresponds to a viscosity parameter in the present embodiment. Hereunder, the index value representing the viscosity of a tissue is also expressed as a viscosity index value.

The index value representing the degree of attenuation of an ultrasonic wave in a tissue to be examined is used, for example, when hepatic steatosis is diagnosed. The index value representing the degree of attenuation of an ultrasonic wave in a tissue to be examined is represented by, for example, an attenuation rate. The attenuation rate takes a larger value, for example, in a case of suffering from hepatic steatosis. The index value representing the degree of attenuation of an ultrasonic wave in a tissue to be examined corresponds to an attenuation parameter in the present embodiment. The index value representing the degree of attenuation of an ultrasonic wave in a tissue to be examined is also expressed as an attenuation index value. The attenuation rate is also referred to as an attenuation constant or an attenuation coefficient.

The degree of variance of a signal reflected from the inside of a tissue of the subject P is, for example, an index value used when hepatic steatosis is diagnosed. The degree of variance of the intensity of the signal reflected from the inside of a tissue of the subject P is represented by, for example, a normalized local variance (NLV) value. The NLV indicates, for example, a degree of matching of a probability density distribution of amplitude values in an echo signal reflected from the liver with respect to the Rayleigh distribution. When it is intended to perform observation from the viewpoint of hepatic steatosis, as a proportion of fat in the liver increases, the B-mode image data becomes a uniform image like a phantom, and the probability density distribution of amplitude values that are the intensity of the echo signal reflected from the liver approaches the Rayleigh distribution, that is, the NLV value approaches 1. When it is intended to perform observation from the viewpoint of hepatic fibrosis, as hepatic fibrosis of the liver progresses, the probability density distribution of amplitude values that are the intensity of the echo signal reflected from the liver reflects a fibrosing structure, which is considered to deviate from the Rayleigh distribution. At this time, the NLV value increases, that is, the NLV value goes away from 1.

The strain ratio is an index value used when hepatic fibrosis is diagnosed. The strain ratio is represented by, for example, a ratio between the elastic modulus of a site of interest and a predetermined elastic modulus serving as a reference. The strain ratio increases as hepatic fibrosis progresses, for example.

The image generation circuitry 35 can also generate three-dimensional B-mode image data by performing coordinate conversion on the three-dimensional B-mode data that is generated by the B-mode processing circuitry 33. The image generation circuitry 35 can also generate three-dimensional color Doppler image data by performing coordinate conversion on the three-dimensional Doppler data that is generated by the Doppler processing circuitry 34.

The image memory 36 is a memory that stores various image data generated by the image generation circuitry 35. The image memory 36 also stores data generated by the B-mode processing circuitry 33 and the Doppler processing circuitry 34. The B-mode data and the Doppler data stored in the image memory 36 can be called by an operator after diagnosis, for example, and becomes display ultrasonic image data via the image generation circuitry 35. The image memory 36 also stores the reflected wave data output from the reception circuitry 32. For example, the image memory 36 is achieved by a random-access memory (RAM), a semiconductor memory element, such as a flash memory, a hard disk, or an optical disc.

The storage 37 stores various data, such as control programs for performing ultrasonic wave transmission and reception, image processing, and display processing, diagnostic protocols, and various body marks. The storage 37 stores predetermined a threshold value for slopes of profiles to be described later and predetermined threshold values for the index values of the tissue characterization parameters, such as an average value of the normalized local variance values. The storage 37 is also used to store data stored in the image memory 36, as necessary. For example, the storage 37 is achieved by a semiconductor memory element, such as a flash memory, a hard disk, or an optical disc.

The processing circuitry 38 executes various data processing. The processing circuitry 38 includes a system control function 381, a display control function 382, an identification function 383, a calculation function 384, and a setting function 385. Here, for example, each processing function of the system control function 381, the display control function 382, the identification function 383, the calculation function 384, and the setting function 385 that are components of the processing circuitry 38 illustrated in FIG. 1, is recorded in the storage 37 in a form of a program executable by a computer. The processing circuitry 38 reads each program from the storage 37 and executes each read program to achieve a function corresponding to each program. In other words, the processing circuitry 38 in a state of reading each program has each function illustrated in the processing circuitry 38 of FIG. 1. The processing circuitry 38 is achieved by, for example, a processor. The system control function 381 is an example of a control unit. The display control function 382 is an example of a display control unit. The identification function 383 is an example of an identification unit. The calculation function 384 is an example of a calculation unit. The setting function 385 is an example of a setting unit.

The system control function 381 is a function of integrally controlling the entire operation of the ultrasonic diagnostic apparatus 1. For example, the system control function 381 controls the transmission circuitry 31 and the reception circuitry 32 based on parameters for transmission and reception of ultrasonic waves according to various modes. Examples of the various modes include a B mode, a Doppler mode, and a tissue characterization measurement mode for measuring each tissue characterization parameter. Examples of the tissue characterization measurement mode include an attenuation imaging (ATI) mode for generating the attenuation image data, an elasticity imaging (shear wave elastography (SWE)) mode for generating the elasticity image data, a viscosity imaging (shear wave dispersion (SWD)) mode for generating the viscosity image data, and a normalized local variance (NLV) mode for generating the variance image data.

The ATI mode is a mode for observing the degree of attenuation of an ultrasonic wave at each position in the depth direction by examining changes in the reflected wave signal in the depth direction when a scatterer distribution in a tissue can be assumed to be uniform. That is, the ATI mode is a mode for observing the attenuation index value among the tissue characterization parameters.

The SWE mode is a mode in which an acoustic radiation force is applied to a living tissue from a body surface by the ultrasonic probe 10 to generate a shear wave, and displacement at each point in a scanning cross section is observed over time. That is, the SWE mode is a mode for observing the elasticity index value among the tissue characterization parameters.

The display control function 382 is a function of controlling the display 70 so as to display ultrasonic images based on various ultrasonic image data generated by the image generation circuitry 35, and the like. For example, the display control function 382 controls the display 70 to display, on the display 70, an image represented by the B-mode image data, an image represented by the color Doppler image data, an image represented by the tissue characterization image data, each piece of the image data being generated by the image generation circuitry 35, or an image including at least two of these images.

The identification function 383 is a function for identifying a target site included in ultrasonic image data of the subject P. The target site identified by the identification function 383 is, for example, a deep portion of the abdominal wall that is the innermost layer of the peritoneum and where a membrane (visceral peritoneum) that covers the liver and a hepatic surface (surface of the liver) are located.

The calculation function 384 is a function of calculating respective index values for a plurality of positions that are specified with the target site serving as a reference, the index values representing reliability of each tissue characterization parameter represented by the ultrasonic image data. Here, the index value representing the reliability of the tissue characterization parameter is, for example, a statistical value, such as an average value, a standard deviation, and a variance of the tissue characterization parameter.

The setting function 385 is a function of setting, based on the results calculated by the calculation function 384, a region of interest for observing the tissue characterization parameter onto an image represented by the ultrasonic image data. The region of interest for observing the tissue characterization parameter is, for example, a region (measurement region of interest (ROI)) for measuring the statistical values of the tissue characterization parameter at respective positions of the subject P. The statistical value of the tissue characterization parameter is an average value, a variance value, or the like of the tissue characterization parameter. The region of interest for observing the tissue characterization parameter may be a region (observation ROI) in which a color map based on the tissue characterization parameter at each position of the subject P is displayed. The color map is an image in which colors or the like mapped from index values representing the tissue characterization parameter are assigned to relevant positions (pixels). The measurement ROI is set into the observation ROI. Hereunder, an imaging ROI is also referred to as the observation ROI or an observation region. The measurement ROI is also referred to as a measurement region hereunder. In the present embodiment, a case where the region of interest for observing the tissue characterization parameter is the observation ROI (imaging ROI) will be described as an example.

In the present embodiment, modes for setting the observation ROI onto an image represented by the ultrasonic image data in the tissue characterization measurement mode include Auto observation ROI and Manual observation ROI. The Auto observation ROI is a mode in which the observation ROI is automatically set onto an image represented by the ultrasonic image data. The Manual observation ROI is a mode in which the observation ROI is manually set onto an image represented by the ultrasonic image data by the user.

The communication circuitry 39 is connected to an external apparatus, such as a server equipped with a picture archiving and communication systems (PACS), and a workstation capable of executing post processing to perform data communication with an external apparatus, for example, via a network.

The input apparatus 50 is an input apparatus for performing various settings and the like and is achieved by, for example, a trackball, a switch button, a mouse, a keyboard, a touchpad that performs an input operation by touching an operation surface, a touchscreen monitor in which a display screen and a touchpad are integrated, non-contact input circuitry using an optical sensor, sound input circuitry, or the like. The input apparatus 50 is connected to the processing circuitry 38 of the apparatus main body 30, converts an input operation accepted from the operator into an electric signal, and outputs the electric signal to the processing circuitry 38 of the apparatus main body 30. Note that, in the present specification, the input apparatus 50 is not limited to the apparatus including physical operating components, such as a mouse and a keyboard. For example, electric signal processing circuitry that accepts an electric signal relevant to an input operation from an external input device provided separately from the apparatus, and outputs the electric signal to the processing circuitry 38 of the apparatus main body 30, is also included in the example of the input apparatus 50.

The display 70 is a display apparatus that displays various ultrasonic images, various settings, and the like. For example, the display 70 displays ultrasonic images based on various ultrasonic image data generated by the apparatus main body 30, a graphical user interface (GUI) for accepting various operations from the user, and the like. In the present embodiment, the display 70 is configured with, for example, a liquid crystal display, a cathode ray tube (CRT) display, or the like.

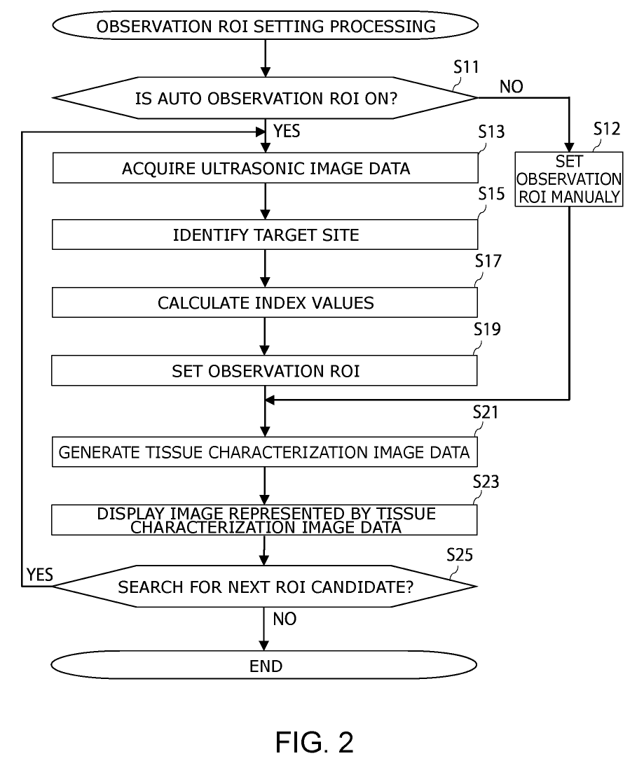

FIG. 2 is a flowchart for explaining observation ROI setting processing executed in the ultrasonic diagnostic apparatus 1 according to the first embodiment. For example, the observation ROI setting processing is processing executed when the ATI mode is started.

As illustrated in FIG. 2, first, the identification function 383 in the processing circuitry 38 of the apparatus main body 30 determines whether the Auto observation ROI is ON (step S11). Specifically, the identification function 383 determines whether the Auto observation ROI is ON by determining whether an input operation for turning ON or OFF of the Auto observation ROI via the input apparatus 50 is accepted.

In step S11, when the Auto observation ROI is ON (step S11: YES), the identification function 383 acquires the ultrasonic image data (step S13). Specifically, the identification function 383 acquires B-mode image data as the ultrasonic image data, from the image generation circuitry 35 or the image memory 36.

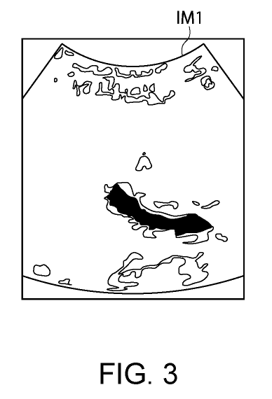

FIG. 3 is a diagram illustrating an example of the B-mode image data according to the first embodiment. In the example illustrated in FIG. 3, the identification function 383 acquires B-mode image data IM1 as the ultrasonic image data.

Next, as illustrated in FIG. 2, the identification function 383 identifies the target site (step S13). Specifically, the identification function 383 identifies the target site from the ultrasonic image data acquired in step S13. The target site is, for example, the deep portion of the abdominal wall where the visceral peritoneum and the hepatic surface are located.

Hereinafter, a method of identifying the target site in the ultrasonic diagnostic apparatus 1 will be described.

In step S15, the identification function 383 first calculates a normalized local variance value by analyzing the B-mode image data IM1 acquired in step S13. Specifically, the identification function 383 calculates a normalized local variance value for each pixel of the B-mode image data IM1. This leads to the identification function 383 generating normalized local variance image data.

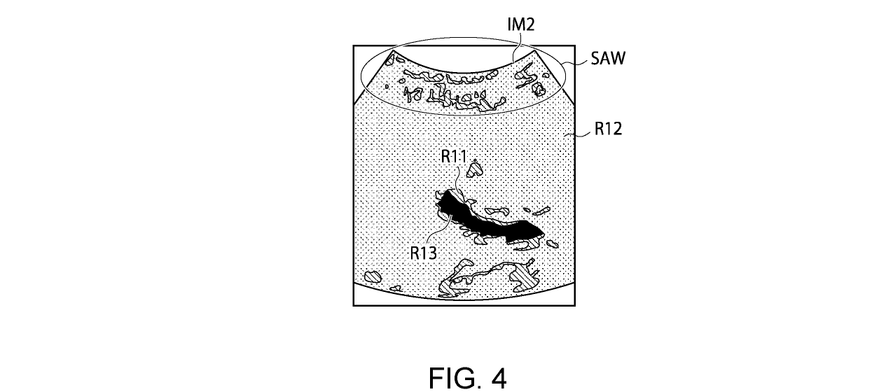

FIG. 4 is a diagram illustrating an example of the normalized local variance image data according to the first embodiment. In the example illustrated in FIG. 4, first regions R11 indicated by hatching extending from upper left to lower right are regions having high normalized local variance values, and are displayed in red, for example. In the example illustrated in FIG. 4, a second region R12 indicated by a dot pattern is a region having low normalized local variance values, and is displayed in blue, for example. In the example illustrated in FIG. 4, a third region R13 that is filled in black is a blackened region, and this blackened region represents, for example, a structure, such as a blood vessel, other than an examination target. In the example illustrated in FIG. 4, a region in the vicinity of the third region R13, that is, a region in the vicinity of the blood vessel includes one of the first regions R11, that is, a region having high normalized local variance values. In a region SAW corresponding to the deep portion of the abdominal wall from under the epidermis illustrated in FIG. 4, the first regions R11 (regions having high normalized local variance values) and the second region R12 (region having low normalized local variance values) coexist.

Next, in step S15, the identification function 383 identifies the deep portion of the abdominal wall included in normalized local variance image data IM2 as the ultrasonic image data. Specifically, the identification function 383 obtains the deep portion of the abdominal wall from results of profiles of the normalized local variance values on a plurality of scanning lines. That is, in the present embodiment, the deep portion of the abdominal wall that is the target site is obtained from the results of the profiles of the normalized local variance values on the plurality of scanning lines.

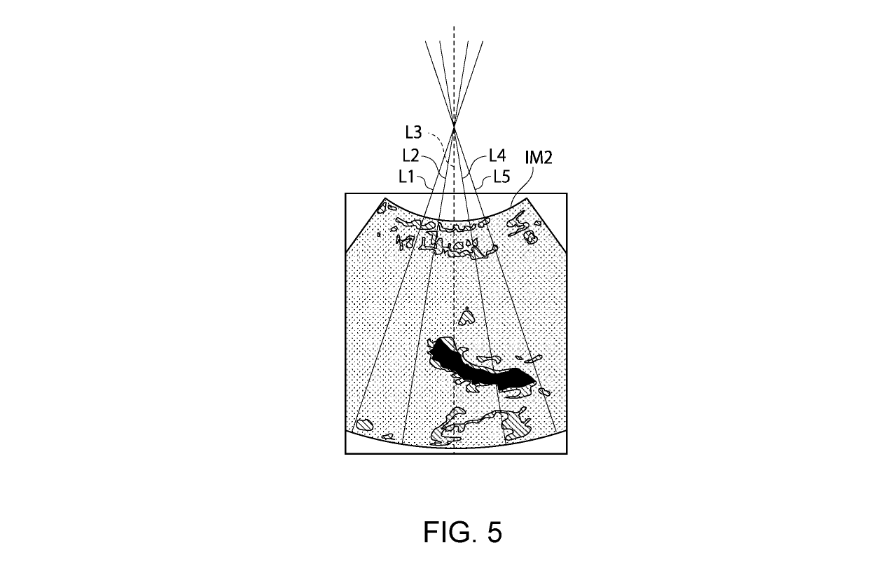

FIGS. 5-7 are diagrams for explaining processing of identifying the target site included in the normalized local variance image data IM2 based on the B-mode image data IM1. First, as illustrated in FIG. 5, the identification function 383 sets the plurality of scanning lines in the normalized local variance image data IM2. In the example illustrated in FIG. 5, the identification function 383 sets a first scanning line L1, a second scanning line L2, a third scanning line L3, a fourth scanning line L4, and a fifth scanning line L5 on the normalized local variance image data IM2 such that intervals between the scanning lines are each, for example, 15 degrees with the third scanning line L3 at the center.

Note that in the example illustrated in FIG. 5, five scanning lines are set on the normalized local variance image data IM2; however, the number of scanning lines set on the normalized local variance image data IM2 is not limited to five. That is, the number of scanning lines set on the normalized local variance image data IM2 is freely selected, and may be two or more and four or less, or six or more. The scanning lines are set on the normalized local variance image data IM2 such that the intervals between the scanning lines are each 15 degrees; however, the intervals between the scanning lines are not limited to 15 degrees. That is, the intervals between the scanning lines are freely selected, and may be 14 degrees or less or 16 degrees or more.

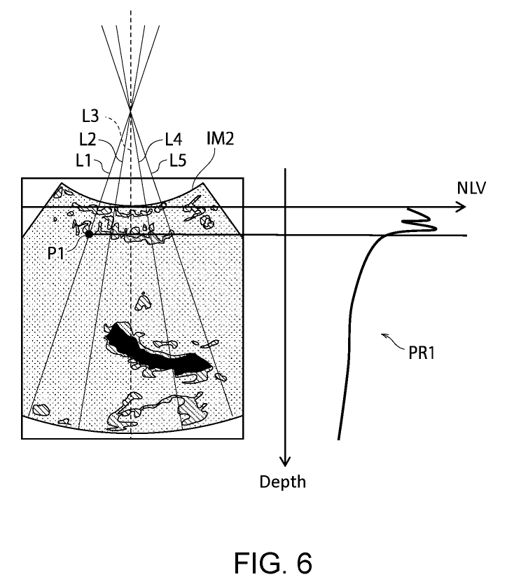

Next, in step S15, the identification function 383 obtains the results (a distribution of the normalized local variance values with respect to transmission directions of a plurality of ultrasonic waves of the subject P (depth direction of the subject P)) of the profiles of the normalized local variance values on the plurality of scanning lines set on the normalized local variance image data IM2. FIG. 6 illustrates a first profile PR1 of the normalized local variance values on the first scanning line L1. In the first profile PR1, the NLV values in the region SAW corresponding to the deep portion of the abdominal wall from under the epidermis illustrated in FIG. 4 are high, and the NLV values are lower at deeper positions. The identification function 383 obtains a position of the target site (deep portion of the abdominal wall) on the first profile PR1, based on the first profile PR1. Specifically, the identification function 383 plots a position where the NLV value in the depth direction of the first profile PR1 is equal to or less than the threshold value, as a position P1 of the target site on the normalized local variance image data IM2. Instead of the position where the NLV value is equal to or less than the threshold value, the identification function 383 may plot a position where a slope indicated by the first profile PR1 is equal to or less than the threshold value, as the position P1 of the target site on the first scanning line L1 on the normalized local variance image data IM2.

Similarly to the first profile PR1, the identification function 383 obtains a position P2 of the target site on the second scanning line L2, a position P3 of the target site on the third scanning line L3, a position P4 of the target site on the fourth scanning line L4, and a position P5 of the target site on the fifth scanning line L5 based on the results of the respective profiles of the second scanning line L2, the third scanning line L3, the fourth scanning line L4, and the fifth scanning line L5.

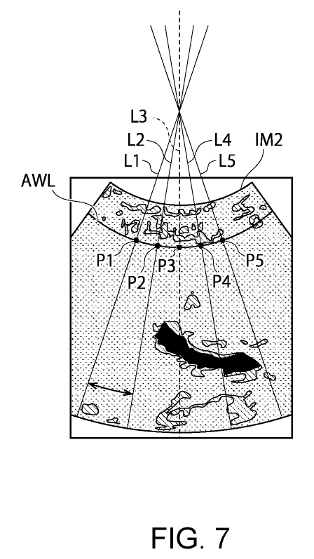

Then, in step S15, the identification function 383 identifies a position of the target site in an azimuth direction based on the position P1 on the first scanning line L1, the position P2 on the second scanning line L2, the position P3 on the third scanning line L3, the position P4 on the fourth scanning line L4, and the position P5 on the fifth scanning line L5. In the example illustrated in FIG. 7, the identification function 383 generates an abdominal wall line AWL having a substantially arc shape obtained by connecting the positions of the target site in the azimuth direction. That is, the identification function 383 generates the abdominal wall line AWL to identify the position of the target site (deep portion of the abdominal wall) in the azimuth direction of the normalized local variance image data IM2.

Next, as illustrated in FIG. 2, the calculation function 384 in the processing circuitry 38 of the apparatus main body 30 calculates the respective index values for a plurality of positions that are specified with the target site identified in step S15 serving as a reference, the index values representing the reliability of the normalized local variance values (tissue characterization parameter) that are represented by the normalized local variance image data IM2 (step S17).

Hereinafter, a method of calculating the index values in the ultrasonic diagnostic apparatus 1 will be described.

In step S17, first, the calculation function 384 acquires observation ROI information. The observation ROI information is information on the observation ROI. The observation ROI information includes, for example, information on a size of the observation ROI and information on a placement position of the observation ROI. The information on the size of the observation ROI includes sizes (lengths) of sides of the observation ROI in the depth direction and sizes (lengths) of sides of the observation ROI in the azimuth direction. The size of the observation ROI is, for example, 2 cm (the lengths of the sides in the azimuth direction) ×4 cm (the lengths of the sides in the depth direction) or more. The information on the placement position of the observation ROI includes, for example, a distance (length) between the abdominal wall line AWL and an upper end of the observation region. The distance (length) between the abdominal wall line AWL and the upper end of the observation region is, for example, the same distance as a thickness from the hypodermis to the hepatic surface or a distance of 1 to 2 cm from the hepatic surface at the upper end of the observation ROI. The observation ROI information is stored in the storage 37.

Next, in step S17, the calculation function 384 sets, to the ultrasonic image data, a plurality of candidate regions for calculating the index values. Specifically, the calculation function 384 sets the plurality of candidate regions to predetermined positions deeper than the deep portion of the abdominal wall in the normalized local variance image data IM2. In other words, the calculation function 384 sets the plurality of candidate regions at positions that avoid the target site (deep portion of the abdominal wall) identified in step S15. That is, the region SAW corresponding to the deep portion of the abdominal wall from under the epidermis is a region affected by multiple reflection of ultrasonic waves (region having a large influence), and is inappropriate for observing the tissue characterization parameter of the subject P. Hence, for example, the calculation function 384 sets the plurality of candidate regions to the normalized local variance image data IM2 such that the distance (length) between the abdominal wall line AWL and the upper end of the observation region is a predetermined length (e.g., a length equivalent to a length from under the epidermis to the deep portion of the abdominal wall) while the size (length of each side) of the observation region based on the observation ROI information is maintained.

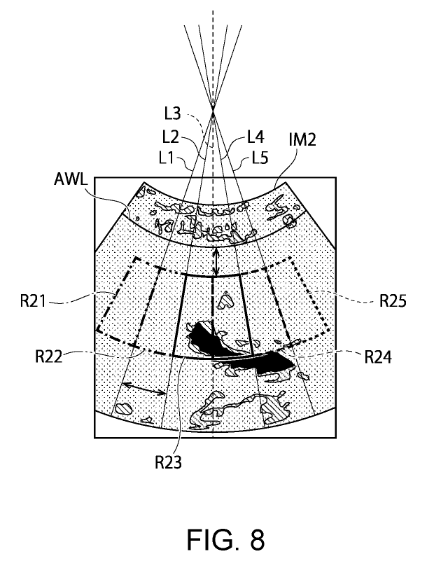

FIG. 8 is a diagram for explaining processing of calculating the index values for the plurality of positions that are specified with the target site serving as the reference in the first embodiment. The calculation function 384 sets candidate regions R21 to R25 to the plurality of positions respectively corresponding to the first scanning line L1 to the fifth scanning line L5 in the normalized local variance image data IM2. In the example illustrated in FIG. 8, widths and lengths of the candidate regions R21 to R25 are the same as the sizes of the observation region (the lengths of the respective sides).

Next, in step S17, the calculation function 384 calculates the respective index values for the plurality of positions that are specified with the target site serving as the reference, the index values representing the reliability of the normalized local variance values (tissue characterization parameter). More specifically, the calculation function 384 specifies the candidate regions R21 to R25 as the plurality of positions, and obtains, as the index values, the statistical values, such as average values of the normalized local variance values in the respective candidate regions R21 to R25.

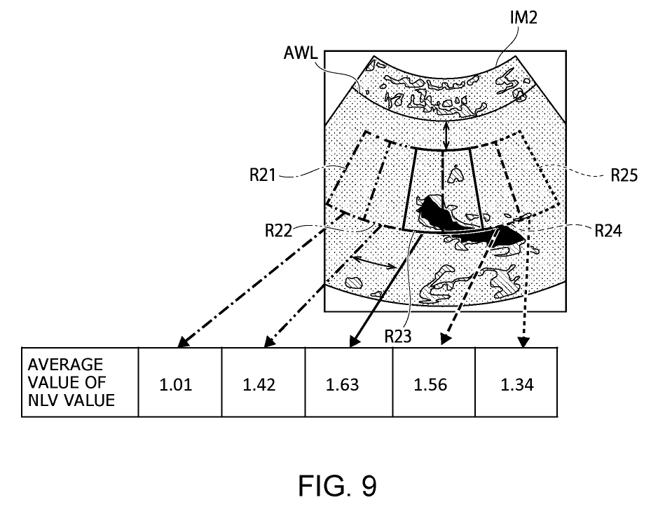

FIG. 9 is a diagram illustrating an example of results of calculating the index values for the plurality of respective positions. As illustrated in FIG. 9, the calculation function 384 calculates, as 1.01, 1.42, 1.63, 1.56, and 1.34, average values of normalized local variance (NLV) values represented by pixels of each of the first candidate region R21, the second candidate region R22, the third candidate region R23, the fourth candidate region R24, and the fifth candidate region R25.

Next, as illustrated in FIG. 2, the setting function 385 in the processing circuitry 38 of the apparatus main body 30 sets the observation ROI (step S19). Specifically, the setting function 385 sets the observation ROI onto the image represented by the B-mode image data IM1 based on the results calculated in step S17. More specifically, the setting function 385 selects one of the candidate regions based on the results calculated in step S17, and sets the selected candidate region as the observation ROI onto the image represented by the B-mode image data IM1. In the present embodiment, the setting function 385 sets the observation ROI onto the image represented by the B-mode image data IM1 while the B-mode image data that is the ultrasonic image data is being reproduced, that is, while a moving image is being displayed.

Hereinafter, a method of setting the observation ROI in the ultrasonic diagnostic apparatus 1 will be described.

First, in step S19, the setting function 385 selects the one of the candidate regions based on the results calculated in step S17. Specifically, the setting function 385 selects, as the one of the candidate regions, a candidate region with an average value of normalized local variance values close to 1 from the average values of the normalized local variance (NLV) values of the pixels included in the plurality of candidate regions, the average values being the results calculated in step S17. This is because the normalized local variance values are values indicating the variances, and a value closer to 1 indicates a more uniform region, and a value farther from 1 indicates a more non-uniform region.

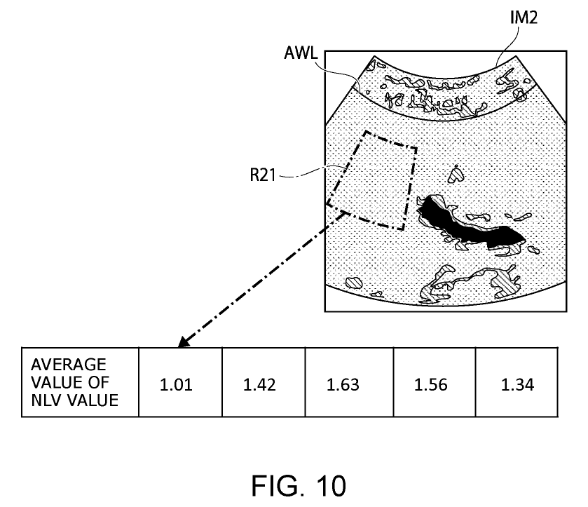

FIG. 10 is a diagram illustrating an example of the selection result according to the first embodiment. As illustrated in FIG. 10, the setting function 385 selects the first candidate region R21 that is a region with an average value of the normalized local variance values closest to 1 among the average values of the normalized local variance (NLV) values of the pixels included in the plurality of candidate regions, the average values being the results calculated in step S17.

Next, in step S19, the setting function 385 sets the observation ROI onto the image represented by the B-mode image data. Specifically, the setting function 385 sets the selected one of the candidate regions as the observation ROI onto the image represented by the B-mode image data.

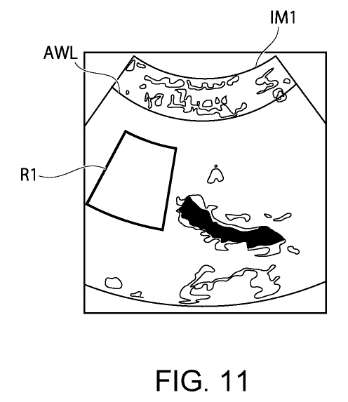

FIG. 11 is a diagram illustrating an example of the region of interest set onto the image represented by the B-mode image data in the first embodiment. As illustrated in FIG. 11, the setting function 385 sets the first candidate region R21 that is the selected one of the candidate regions, as an observation region R1 onto the image represented by the B-mode image data IM1.

Next, as illustrated in FIG. 2, the display control function 382 generates the tissue characterization image data (step S21). Specifically, the display control function 382 controls the image generation circuitry 35 to generate the attenuation image data. More specifically, since the ATI mode is performed, the display control function 382 causes the image generation circuitry 35 to obtain an attenuation constant for each pixel included in the observation ROI set in step S19 and generate the attenuation image data in which a pixel value relevant to the attenuation constant is assigned to each pixel.

Next, as illustrated in FIG. 2, the display control function 382 displays an image represented by the tissue characterization image data (step S23). Specifically, the display control function 382 displays the image represented by the attenuation image data generated in step S21, as the image represented by the tissue characterization image data.

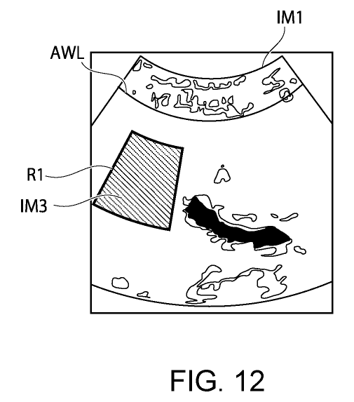

FIG. 12 is a diagram illustrating an example of the image represented by the tissue characterization image data according to the first embodiment. As illustrated in FIG. 12, an image represented by attenuation image data IM3 is displayed in the observation region R1 so as to be superimposed on the image represented by the B-mode image data IM1.

Next, as illustrated in FIG. 2, the identification function 383 determines whether a next ROI candidate is searched for (step S25). Specifically, the identification function 383 determines whether the next ROI candidate is searched for by determining whether an input operation for turning ON or OFF of the Auto observation ROI via the input apparatus 50 is accepted. In step S25, when the next ROI candidate is searched for (step S25: YES), the processing returns to step S13, and the processing from step S13 is repeated and waits.

On the other hand, in step S11, when the Auto observation ROI is not ON (step S11: NO), the identification function 383 determines whether a user operation related to setting of an observation ROI is accepted. When the user operation related to the setting of the observation ROI is not accepted, the processing returns to step S11 and the Auto observation ROI is turned ON, then, or until the user operation related to the setting of the observation ROI is accepted, the processing from step S11 is repeated. On the other hand, when the user operation related to the setting of the observation ROI is accepted, the setting function 385 sets an observation ROI based on the user operation related to the setting of the observation ROI (step S12). That is, in step S12, the observation ROI is set manually. Next, as in steps S21 and S23, the display control function 382 generates tissue characterization image data and displays an image represented by the tissue characterization image data.

When the next ROI candidate is not searched for in step S25 (step S25: NO), the observation ROI setting processing ends, or when the Auto observation ROI is not ON in step S11, a user operation related to setting of an observation ROI is accepted in step S12, the display control function 382 displays an image represented by tissue characterization image data in step S23, and then the observation ROI setting processing ends.

As described above, in the ultrasonic diagnostic apparatus 1, the deep portion of the abdominal wall that is the target site included in the ultrasonic image data of the subject P is identified as the target site, the respective index values (average values) representing the reliability of the normalized local variance values are calculated for the plurality of candidate regions that are specified with the deep portion of the abdominal wall serving as the reference, and the observation ROI is set onto the image represented by the B-mode image data based on the calculated results. This allows the observation ROI to be set at a position with a few structures, so that the tissue characterization of the subject P can be analyzed with high accuracy.

Note that, in the first embodiment described above, the observation ROI setting processing is described as processing executed when the ATI mode is started; however, the mode is not limited to the ATI mode. For example, the observation ROI setting processing may be processing executed when the tissue characterization measurement mode, such as the SWE mode, is started.

### First Modification

In a First Embodiment, after execution of the observation ROI setting processing, an ultrasonic diagnostic apparatus 1 may cause the user to set a measurement ROI into the observation ROI set to the ultrasonic image data IM1 by displaying information based on the index value indicating the reliability of the tissue characterization parameter for each position in the observation ROI.

Specifically, first, a calculation function 384 obtains a statistical value, such as a standard deviation and a variance, as the index value representing the reliability of the attenuation constant (tissue characterization parameter), for each position in the observation ROI set to the ultrasonic image data IM1. For example, the calculation function 384 obtains a standard deviation of the attenuation constant for each pixel or each pixel group of the observation ROI. Next, a display control function 382 generates, as identification information, information (a distribution image of the standard deviations) in which a pixel value (color information) mapped to the standard deviation obtained for each pixel or each pixel group is allocated to each position (pixel or pixel group) in the observation ROI, or information (an image indicating a region) indicating a position where the standard deviation in the observation ROI is equal to or larger than a threshold value, and displays the generated identification information in the observation ROI.

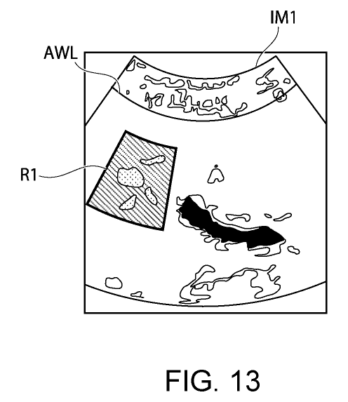

FIG. 13 is a diagram illustrating an example of the identification information displayed in the observation ROI. In the example illustrated in FIG. 13, the display control function 382 displays the information indicating the distribution of the standard deviations, in an observation region R1 as the identification information. Regions indicated by a dot pattern in the observation region R1 are regions with high standard deviations, and a region indicated by hatching extending from upper left to lower right in the observation region R1 is a region with low standard deviations. Since the user can set the measurement ROI while visually checking the identification information by displaying the identification information in the observation region R1 in this manner, the measurement ROI can be easily set to a region with low standard deviations, that is, a region representing a structure being an examination target, such as the liver, while avoiding a region with high standard deviations, that is, a region representing a structure, such as a blood vessel, other than an examination target.

### Second Embodiment

In the first embodiment, the case where the ultrasonic diagnostic apparatus 1 sets the observation ROI to the ultrasonic image data IM1 as the region of interest for observing the tissue characterization parameter is described; however, the region of interest for observing the tissue characterization parameter set to the ultrasonic image data IM1 is not limited to the observation ROI. In a second embodiment, for example, a case where an ultrasonic diagnostic apparatus 1 sets, as the region of interest for observing the tissue characterization parameter, a measurement ROI in ultrasonic image data IM1 instead of the observation ROI will be described. Since the configuration of the ultrasonic diagnostic apparatus 1 is the same as that of the first embodiment described above, the description thereof will be omitted.

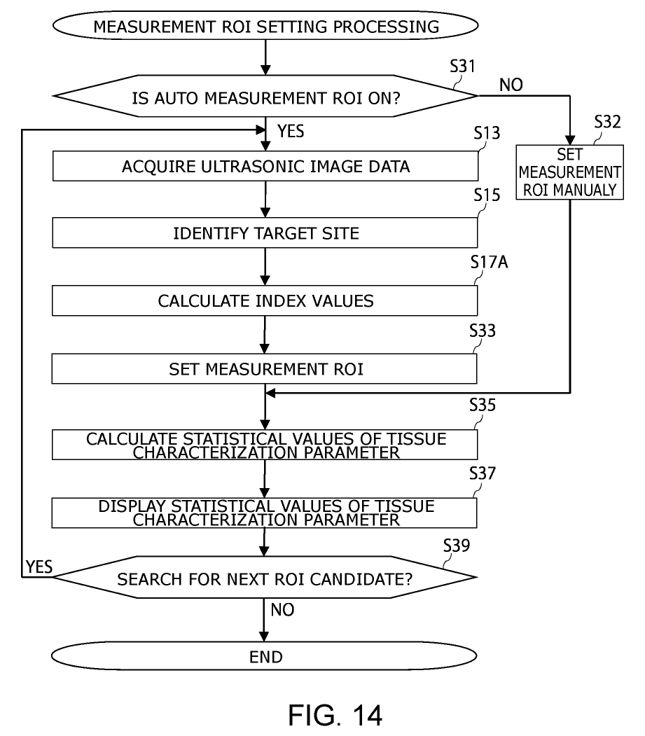

FIG. 14 is a flowchart for explaining measurement ROI setting processing executed in the ultrasonic diagnostic apparatus 1 according to the second embodiment. For example, the measurement ROI setting processing is processing executed when the ATI mode is started.

As illustrated in FIG. 14, first, an identification function 383 in processing circuitry 38 of an apparatus main body 30 determines whether Auto measurement ROI is ON (step S31). Specifically, the identification function 383 determines whether the Auto measurement ROI is ON by determining whether an input operation for turning ON or OFF of the Auto measurement ROI via an input apparatus 50 is accepted.

In step S31, when the Auto observation ROI is ON (step S31: YES), the identification function 383 acquires the ultrasonic image data (step S13). Note that the processing from step S13 to step S15 is the same as that in FIG. 2, and thus the description thereof will be omitted.

Next, as illustrated in FIG. 14, a calculation function 384 in the processing circuitry 38 of the apparatus main body 30 calculates respective index values for a plurality of positions that are specified with the target site identified in step S15 serving as the reference, the index values representing the reliability of the normalized local variance values (tissue characterization parameter) that are represented by the normalized local variance image data IM2 (step S17A).

Hereinafter, a method of calculating the index values in the ultrasonic diagnostic apparatus 1 will be described.

In step S17A, first, the calculation function 384 acquires measurement ROI information. The measurement ROI information is information on the measurement ROI. The measurement ROI information includes, for example, information on a size of the measurement ROI and information on a placement position of the measurement ROI. The information on the size of the measurement ROI includes sizes (lengths) of sides of the measurement ROI in the depth direction and sizes (lengths) of sides of the measurement ROI in the azimuth direction. The measurement ROI has, for example, such size that the lengths of the sides in the azimuth direction are the same as the lengths of the observation ROI in the azimuth direction, and the lengths of the sides in the depth direction are 4 cm or more. The information on the placement position of the measurement ROI includes a placement position at an upper end of the measurement ROI. The placement position at the upper end of the measurement ROI is, for example, a boundary between a region where the upper end of the measurement ROI is affected by multiple reflection and a region not affected by the multiple reflection. The boundary between the region affected by the multiple reflection and the region not affected by the multiple reflection is in the vicinity of the target site (deep portion of the abdominal wall). The measurement ROI information is stored in storage 37.

The processing, performed by the calculation function 384, from the setting of the plurality of candidate regions to the calculation of the respective index values representing the reliability of the respective normalized local variance values (tissue characterization parameter) for the plurality of positions that are specified with the target site serving as the reference, is similar to that of the first embodiment described above, and thus description thereof is omitted.

Next, as illustrated in FIG. 14, a setting function 385 in the processing circuitry 38 of the apparatus main body 30 sets the measurement ROI (step S33). Specifically, the setting function 385 sets the measurement ROI onto the image represented by the B-mode image data IM1 based on the results calculated in step S17A. More specifically, the setting function 385 selects one of the candidate regions based on the results calculated in step S17A, and sets the selected candidate region as the measurement ROI onto the image represented by the B-mode image data IM1. In the present embodiment, the setting function 385 sets the measurement ROI onto the image represented by the B-mode image data IM1 while the B-mode image data that is the ultrasonic image data is being reproduced, that is, while the moving image is being displayed. Since the method of setting the measurement ROI is similar to the method of setting the observation ROI according to the first embodiment described above, the description thereof will be omitted.

Next, as illustrated in FIG. 14, the calculation function 384 calculates statistical values of the tissue characterization parameter in the measurement ROI (step S35). Specifically, since the ATI mode is performed, the calculation function 384 calculates the statistical values of the attenuation constant in the measurement ROI as the statistical values of the tissue characterization parameter in the measurement ROI.

Next, as illustrated in FIG. 14, a display control function 382 displays the statistical values of the tissue characterization parameter (step S37). Specifically, the display control function 382 displays the statistical values of the tissue characterization parameter calculated in step S35 on a display 70. Specifically, the display control function 382 displays the statistical values, calculated in step S35, of the attenuation constant in the measurement ROI on the display 70.

Next, as illustrated in FIG. 14, the identification function 383 determines whether the next ROI candidate is searched for (step S39). Specifically, the identification function 383 determines whether the next ROI candidate is searched for by determining whether an input operation for turning ON or OFF of the Auto measurement ROI via the input apparatus 50 is accepted. In step S39, when the next ROI candidate is searched for (step S39: YES), the processing returns to step S13, and the processing from step S13 is repeated and waits.

On the other hand, in step S31, when the Auto measurement ROI is not ON (step S31: NO), the identification function 383 determines whether a user operation related to setting of a measurement ROI is accepted. When the user operation related to the setting of the measurement ROI is not accepted, the processing returns to step S31 and the Auto measurement ROI is turned ON, then, or until the user operation related to the setting of the measurement ROI is accepted, the processing from step S31 is repeated. On the other hand, when the user operation related to the setting of the measurement ROI is accepted, the setting function 385 sets a measurement ROI based on the user operation related to the setting of the measurement ROI (step S32). That is, in step S32, the measurement ROI is set manually. Next, as in steps S35 and S37, the calculation function 384 calculates statistical values of the tissue characterization parameter and displays the statistical values of the tissue characterization parameter.

When the next ROI candidate is not searched for in step S39 (step S39: NO), the measurement ROI setting processing ends, or when the Auto measurement ROI is not ON in step S31, a user operation related to setting of a measurement ROI is accepted in step S32, statistical values of the tissue characterization parameter are displayed in step S37, and then the measurement ROI setting processing ends.

As described above, in the ultrasonic diagnostic apparatus 1, the deep portion of the abdominal wall that is the target site included in the ultrasonic image data of the subject P is identified as the target site, the respective average values of the normalized local variance values representing the reliability of the normalized local variance values that represent the ultrasonic image data (normalized local variance image data) are calculated for the plurality of candidate regions that are specified with the deep portion of the abdominal wall serving as the reference, and the measurement ROI is set onto the image represented by the B-mode image data based on the calculated results. This allows the measurement ROI to be set at a position with a few structures, so that the tissue characterization of the subject P can be analyzed with high accuracy.

In the second embodiment, the case where the ultrasonic diagnostic apparatus 1 sets the measurement ROI, instead of the observation ROI, to the ultrasonic image data IM1 as the region of interest for observing the tissue characterization parameter is described; however, the measurement ROI set to the ultrasonic image data IM1 may be used as the observation ROI. For example, the ultrasonic diagnostic apparatus 1 may use the measurement ROI as the observation ROI by accepting an input operation for switching the measurement ROI set to the ultrasonic image data IM1 to the observation ROI. In this case, the ultrasonic diagnostic apparatus 1 results in displaying the statistical values of the tissue characterization parameter in the measurement ROI when the measurement ROI is used, and displaying an image represented by the tissue characterization image data in the measurement ROI used as the observation ROI when the measurement ROI is used as the observation ROI.

### Third Embodiment

In the ultrasonic diagnostic apparatus 1 according to the first and second embodiments described above, the case where either of the observation ROI or the measurement ROI is set to the ultrasonic image data IM1 is described; however, the region of interest for observing the tissue characterization parameter set to the ultrasonic image data IM1 is not limited to the observation ROI and the measurement ROI. In a third embodiment, a case where an ultrasonic diagnostic apparatus 1 sets, as the region of interest for observing the tissue characterization parameter, both the observation ROI and the measurement ROI to the ultrasonic image data IM1 will be described. Since the configuration of the ultrasonic diagnostic apparatus 1 is the same as that of the first embodiment described above, the description thereof will be omitted.

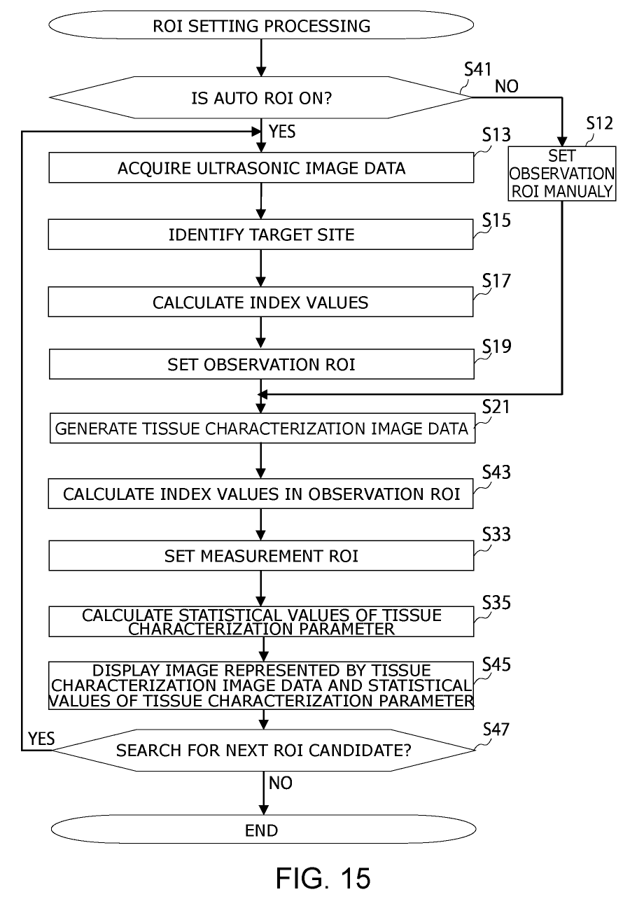

FIG. 15 is a flowchart for explaining ROI setting processing executed in the ultrasonic diagnostic apparatus 1 according to the third embodiment. For example, the ROI setting processing is processing executed when the ATI mode is started.

As illustrated in FIG. 15, first, an identification function 383 in processing circuitry 38 of an apparatus main body 30 determines whether Auto ROI is ON (step S41). Specifically, the identification function 383 determines whether the Auto ROI is ON by determining whether an input operation for turning ON or OFF of the Auto ROI via an input apparatus 50 is accepted.

In step S41, when the Auto ROI is ON (step S41: YES), the identification function 383 acquires the ultrasonic image data (step S13). Note that the processing from step S13 to step S21 is the same as that in FIG. 2, and thus the description thereof will be omitted.

Next, as illustrated in FIG. 15, a calculation function 384 in the processing circuitry 38 of the apparatus main body 30 calculates the respective index values for the plurality of positions in the observation ROI (step S43). Specifically, the calculation function 384 calculates respective index values that are specified with the target site, as the reference, identified in step S15 and that, for the plurality of positions in the observation ROI, represent the reliability of the normalized local variance values (tissue characterization parameter) represented by the normalized local variance image data IM2.

Hereinafter, a method of calculating the index values in the ultrasonic diagnostic apparatus 1 will be described.

In step S43, as in step S17A in the second embodiment, the calculation function 384 acquires measurement ROI information.

Next, in step S43, the calculation function 384 sets, to the ultrasonic image data, a plurality of candidate regions for calculating the index values. Specifically, the calculation function 384 sets the plurality of candidate regions to predetermined positions that are set to the normalized local variance image data IM2 and are deeper than the deep portion of the abdominal wall in the observation ROI. For example, the calculation function 384 sets the plurality of candidate regions with a size of a measurement region based on the measurement ROI information, to the normalized local variance image data IM2 such that upper ends of the plurality of candidate regions are located in the vicinity of the abdominal wall line AWL in the observation ROI.

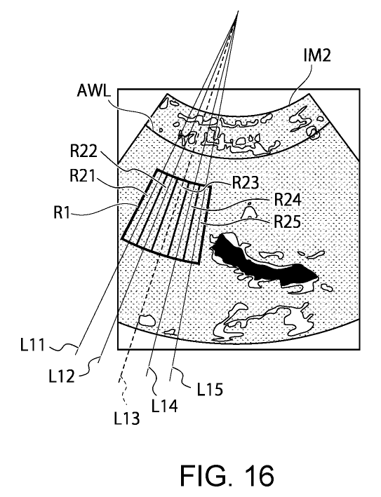

FIG. 16 is a diagram for explaining processing of calculating the index values for the plurality of positions that are specified with the target site serving as the reference in the third embodiment. The calculation function 384 sets candidate regions R21 to R25 to the plurality of positions respectively corresponding to the first scanning line L11 to the fifth scanning line L15 in the normalized local variance image data IM2. In the example illustrated in FIG. 16, widths and lengths of the candidate regions R21 to R25 are the same as the sizes of the observation region (the lengths of the respective sides). The processing of step S33 and step S35 after step S43 is the same as that in the second embodiment, and thus the description thereof will be omitted.

Next, as illustrated in FIG. 15, a display control function 382 displays the tissue characterization image data and the statistical values of the tissue characterization parameter (step S45). Specifically, the display control function 382 displays, on a display 70, the image represented by the tissue characterization image data and the statistical values of the tissue characterization parameter calculated in step S35. More specifically, since the ATI mode is performed, the display control function 382 displays the image represented by the attenuation image data generated in step S21 and the statistical values, calculated in step S35, of the attenuation constant in the measurement ROI.

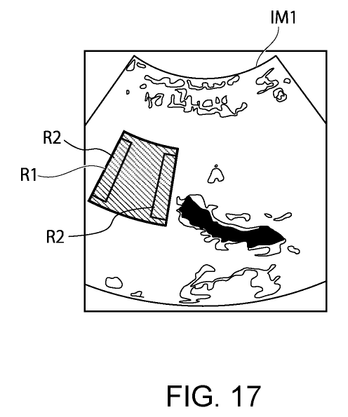

FIG. 17 is a diagram illustrating an example of the measurement ROIs and the observation ROI that are set onto the image represented by the B-mode image data in the third embodiment. As illustrated in FIG. 17, a setting function 385 sets the first candidate region R21 that is the selected one of the candidate regions and the fifth candidate region R25, as measurement regions R2 onto the image represented by the B-mode image data IM1. Then, the image represented by the attenuation image data generated in step S21 is displayed in the observation region R1 so as to be superimposed on the image represented by the B-mode image data IM1.

Next, as illustrated in FIG. 15, an identification function 383 determines whether the next ROI candidate is searched for (step S47). Specifically, the identification function 383 determines whether the next ROI candidate is searched for by determining whether an input operation for turning ON or OFF of the Auto ROI via the input apparatus 50 is accepted. In step S47, when the next ROI candidate is searched for (step S47: YES), the processing returns to step S13, and the processing from step S13 is repeated and waits.

On the other hand, in step S41, when the Auto ROI is not ON (step S41: NO), the identification function 383 determines whether a user operation related to setting of an observation ROI is accepted. When the user operation related to the setting of the observation ROI is not accepted, the processing returns to step S41 and the Auto ROI is turned ON, then, or until the user operation related to the setting of the observation ROI is accepted, the processing from step S41 is repeated. On the other hand, when the user operation related to the setting of the observation ROI is accepted, the setting function 385 sets an observation ROI based on the user operation related to the setting of the observation ROI (step S12). That is, in step S12, the observation ROI is set manually. The processing in steps later than step S12 is the same as the processing in the above-described steps S21 to S47, and thus, the description thereof will be omitted. Alternatively, the measurement ROI may be manually set together with the observation ROI. In this case, after step S12, as in steps S21 and S23, the display control function 382 generates tissue characterization image data and displays an image represented by the tissue characterization image data. Next, the identification function 383 determines whether a user operation related to setting of a measurement ROI is accepted. When a user operation related to setting of a measurement ROI is not accepted, the processing waits until the user operation related to the setting of the measurement ROI is accepted. On the other hand, when the user operation related to setting of the measurement ROI is accepted, the setting function 385 sets a measurement ROI based on the user operation related to the setting of the measurement ROI. Next, as in steps S35 and S37, the calculation function 384 calculates statistical values of the tissue characterization parameter and displays the statistical values of the tissue characterization parameter.

When the next ROI candidate is not searched for in step S47 (step S47: NO), the ROI setting processing ends, or when the Auto ROI is not ON in step S41, a user operation related to setting of a measurement ROI is accepted in step S12, the statistical values of the tissue characterization parameter are displayed in step S45, and then the ROI setting processing ends.

As described above, the ultrasonic diagnostic apparatus 1 identifies, as the target site, the deep portion of the abdominal wall that is the target site included in the ultrasonic image data of the subject P, and calculates the respective index values (average values (statistical values)) for the plurality of candidate regions that are specified with the deep portion of the abdominal wall serving as the reference, the index values representing the reliability of the normalized local variance values represented by the ultrasonic image data (normalized local variance image data). The ultrasonic diagnostic apparatus 1 sets the observation ROI onto the image represented by the B-mode image data based on the results calculated for the plurality of candidate regions. This allows the observation ROI to be set at a position with a few structures and the measurement ROIs to be set at positions each with a few structures in the observation ROI, so that the tissue characterization of the subject P can be analyzed with high accuracy.

### Second Modification

In the ultrasonic diagnostic apparatus 1 according to the second and third embodiments described above, the setting function 385 sets the measurement region(s) R2 onto the image represented by the ultrasonic image data while the ultrasonic image data is being reproduced, that is, while the moving image is being displayed; however, the measurement ROI can also be set onto the image represented by the ultrasonic image data while the reproduction of the ultrasonic image data is stopped, that is, while the moving image display is stopped. Hereinafter, a case where this modification is applied to the second embodiment will be described as a second modification, and portions different from those of the second embodiment described above will be described. Since the configuration of an ultrasonic diagnostic apparatus 1 is the same as that of the second embodiment described above, that is, the same as that of FIG. 1 in the first embodiment, the description thereof will be omitted.

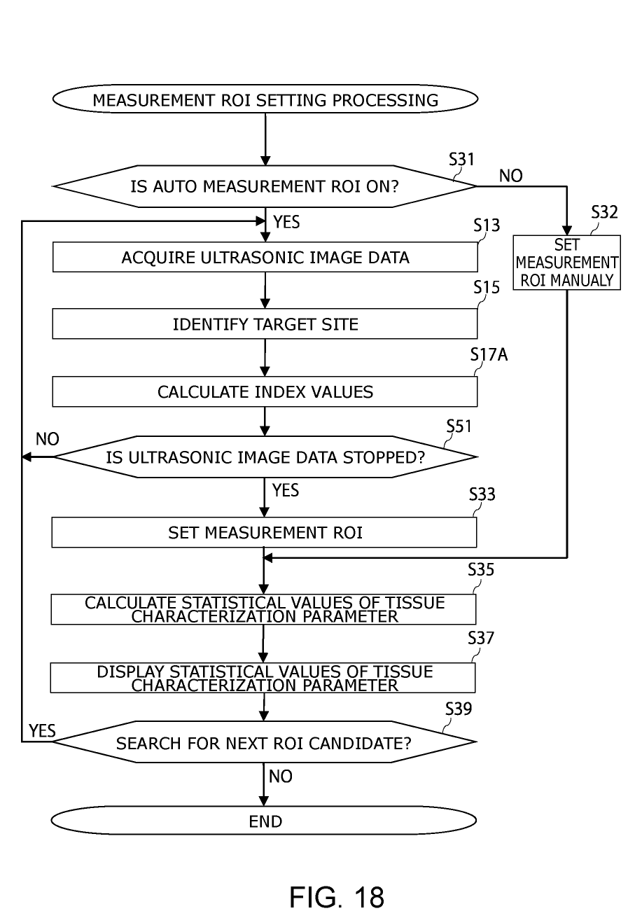

FIG. 18 is a flowchart for explaining measurement ROI setting processing executed in the ultrasonic diagnostic apparatus according to the second modification, and the measurement ROI setting processing relevant to FIG. 14 is, for example, processing executed when the ATI mode is started. The processing in steps S13 to S15 illustrated in FIG. 18 is the same as that in FIG. 2, and the processing in steps S31 and S17A is the same as that in FIG. 14, and thus description thereof is omitted.

Next, as illustrated in FIG. 18, a setting function 385 in processing circuitry 38 of an apparatus main body 30 determines whether the ultrasonic image data is stopped (step S51). Specifically, the setting function 385 determines whether an image represented by the ultrasonic image data displayed on a display 70 is stopped by determining whether an input operation for stopping the image represented by the ultrasonic image data via an input apparatus 50 is accepted. When the ultrasonic image data is not stopped (Step S51: NO), the processing returns to Step S13, and the processing from Step S13 is repeated and waits.

On the other hand, in step S51, when the ultrasonic image data is stopped (step S51: YES), the setting function 385 sets the measurement ROI (step S33). That is, the setting function 385 sets the measurement ROI into the observation ROI when the reproduction of the ultrasonic image data is stopped. The processing in steps S33 to S39 after the step S33 is the same as that in the second embodiment described above, and thus, the description thereof will be omitted. The processing in step S32 is the same as that in the second embodiment described above, and thus, the description thereof will be omitted.

When the next ROI candidate is not searched for in step S39 (step S39: NO), the measurement ROI setting processing ends, or when the Auto measurement ROI is not ON in step S31, a user operation related to setting of a measurement ROI is accepted in step S32, statistical values of the tissue characterization parameter are displayed in step S37, and then the measurement ROI setting processing ends.

As described above, in the ultrasonic diagnostic apparatus 1 according to the second modification, the setting function 385 sets the measurement region onto the image represented by the ultrasonic image data while the reproduction of the ultrasonic image data is stopped, that is, while the moving image display is stopped, and thus the measurement ROI can be set into the observation ROI on an image that is represented by the ultrasonic image data and intended by the user.

### Fourth Embodiment

In the first to third embodiments, the first modification, and the second modification described above, the case where the ultrasonic diagnostic apparatus 1 executes various processing is described; however, a medical image processing apparatus may execute processing similar to the processing executed by the ultrasonic diagnostic apparatus 1. In the following, by reference to such an embodiment as a fourth embodiment, portions different from those of the first embodiment will be described. In the following, while a case where this modification is applied to the first embodiment will be described, this modification can also be applied to the second embodiment, the third embodiment, the first modification, and the second modification described above.

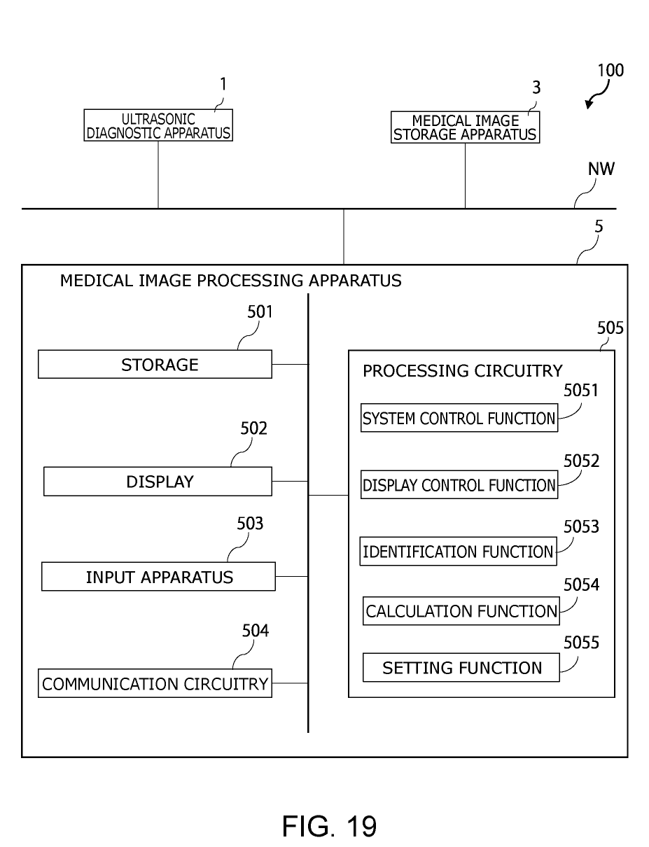

FIG. 19 is a block diagram illustrating a configuration example of a medical image processing system according to the fourth embodiment. As illustrated in FIG. 19, a medical image processing system 100 includes an ultrasonic diagnostic apparatus 1, a medical image storage apparatus 3, and a medical image processing apparatus 5. As illustrated in FIG. 19, the ultrasonic diagnostic apparatus 1, the medical image storage apparatus 3, and the medical image processing apparatus 5 are communicatively connected to one another via a network NW.

The ultrasonic diagnostic apparatus 1 generates ultrasonic image data. The ultrasonic diagnostic apparatus 1 transmits the generated ultrasonic image data to the medical image processing apparatus 5 via communication circuitry 39.

The medical image storage apparatus 3 stores the ultrasonic image data generated by the ultrasonic diagnostic apparatus 1. For example, the medical image storage apparatus 3 is achieved by a computer device, such as a server apparatus. The medical image storage apparatus 3 acquires the ultrasonic image data from the ultrasonic diagnostic apparatus 1 via the network NW, and stores the acquired ultrasonic image data in a memory, such as a hard disk or an optical disc provided inside or outside the apparatus. In addition, the medical image storage apparatus 3 transmits the ultrasonic image data stored in the memory to the medical image processing apparatus 5 in response to a request from the medical image processing apparatus 5.

The medical image processing apparatus 5 acquires the ultrasonic image data from the ultrasonic diagnostic apparatus 1 or the medical image storage apparatus 3 via the network NW, and executes processing, on the acquired ultrasonic image data, similar to the processing executed by the ultrasonic diagnostic apparatus 1 according to the first or second embodiment. For example, the medical image processing apparatus 5 executes observation ROI setting processing to acquire ultrasonic image data, identify a target site, calculate index values, and set an observation ROI. The medical image processing apparatus 5 executes measurement ROI setting processing to acquire ultrasonic image data with the observation ROI set, identify a target site, calculate index values, and set a measurement ROI. As illustrated in FIG. 19, the medical image processing apparatus 5 includes storage 501, a display 502, an input apparatus 503, communication circuitry 504, and processing circuitry 505.

The storage 501 stores various data, such as control programs for performing ultrasonic wave transmission and reception, image processing, and display processing. The storage 501 is a memory that stores various images acquired from the ultrasonic diagnostic apparatus 1 or the medical image storage apparatus 3. For example, the storage 501 is achieved by a semiconductor memory element, such as a flash memory, a hard disk, or an optical disc.

The display 502 displays a graphical user interface (GUI) for the user to input various setting requests using the input apparatus 503, and displays an image represented by the ultrasonic image data. The display 502 displays various messages in order to notify the user of a processing status.

The input apparatus 503 is connected to the processing circuitry 505, converts an input operation accepted from the user into an electric signal, and outputs the electric signal to the processing circuitry 505. Note that the input apparatus 503 is not limited to the apparatus including physical operating components, such as a mouse and a keyboard. For example, processing circuitry that accepts an electric signal relevant to an input operation from an external input device provided separately from the apparatus, and outputs the electric signal to control circuitry is also included in the example of the input apparatus. The input apparatus 503 is achieved by, for example, a trackball, a switch button, a mouse, a keyboard, a touchpad that performs an input operation by touching an operation surface, a touchscreen in which a display screen and a touchpad are integrated, a non-contact input apparatus using an optical sensor, a sound input apparatus, or the like.

The communication circuitry 504 is connected to the processing circuitry 505, and controls transmission of various data between the communication circuitry 504 and the ultrasonic diagnostic apparatus 1 connected via the network NW and communication with the ultrasonic diagnostic apparatus 1. For example, the communication circuitry 504 is achieved by a network card, a network adapter, a network interface controller (NIC), or the like.

The processing circuitry 505 executes various data processing. The processing circuitry 505 includes a system control function 5051, a display control function 5052, an identification function 5053, a calculation function 5054, and a setting function 5055. Here, for example, each processing function of the system control function 5051, the display control function 5052, the identification function 5053, the calculation function 5054, and the setting function 5055 that are components of the processing circuitry 505 illustrated in FIG. 19, is recorded in the storage 501 in a form of a program executable by a computer. The processing circuitry 505 reads each program from the storage 501 and executes each read program to achieve a function corresponding to each program. In other words, the processing circuitry 505 in a state of reading each program has each function illustrated in the processing circuitry 505 of FIG. 19. The processing circuitry 505 is achieved by, for example, a processor.

The system control function 5051 corresponds to the system control function 381 described above. The system control function 5051 performs processing similar to that of the system control function 381. The system control function 5051 is an example of the control unit. The display control function 5052 corresponds to the display control function 382 described above. The display control function 5052 performs processing similar to that of the display control function 382. The display control function 5052 is an example of the display control unit. The identification function 5053 corresponds to the identification function 383 described above. The identification function 5053 performs processing similar to that of the identification function 383. The identification function 5053 is an example of the identification unit. The calculation function 5054 corresponds to the calculation function 384 described above. The calculation function 5054 performs processing similar to that of the calculation function 384. The calculation function 5054 is an example of the calculation unit. The setting function 5055 corresponds to the setting function 385 described above. The setting function 5055 performs processing similar to that of the setting function 385. The setting function 5055 is an example of the setting unit.

As described above, also in the medical image processing apparatus 5 in the medical image processing system 100 according to the fourth embodiment, the tissue characterization of the subject can be analyzed with high accuracy similarly to the ultrasonic diagnostic apparatus 1 described above.

The medical image processing apparatus 5 according to the fourth embodiment described above may be achieved by a workstation or cloud computing. At this time, the display 502 and the input apparatus 503 may be connected to a network as client apparatuses, for example. The storage 501, the communication circuitry 504, and the processing circuitry 505 may be mounted on a server on the network.

In the medical image processing system 100 according to the fourth embodiment described above, the case where the ultrasonic diagnostic apparatus 1 is connected to the network NW is described; however, a medical image diagnostic apparatus connected to the network NW is not limited to the ultrasonic diagnostic apparatus. That is, the medical image diagnostic apparatus connected to the network NW is freely selected, and may be, for example, an X-ray diagnostic apparatus, such as a general X-ray diagnostic apparatus and an X-ray computed tomography (CT) apparatus, a magnetic resonance imaging (MRI) apparatus, a single photon emission computed tomography (SPECT) apparatus, a positron emission computed tomography (PET) apparatus, or the like. That is, the medical image processing apparatus 5 in the medical image processing system 100 may execute processing similar to various processing executed by the medical image processing apparatus 5 on medical image data, such as X-ray image data, MRI image data, SPECT image data, and PET image data, acquired from the above medical image diagnostic apparatus or the medical image storage apparatus 3.

### Other Modifications

In the third embodiment described above, the case where the ultrasonic diagnostic apparatus 1 automatically sets each of the observation ROI and the measurement ROI to the ultrasonic image data IM1 is described; however, the measurement ROI setting processing according to the second embodiment may be executed to automatically set the measurement ROI into the observation ROI after the user sets the observation ROI onto the image represented by the ultrasonic image data IM1 by manual operation via the input apparatus 50.

In the ultrasonic diagnostic apparatus 1 according to the first to fourth embodiments, the first modification, and the second modification, the case where the target site is identified using the NLV values is described; however, this is not intended to be limiting. For example, the ultrasonic diagnostic apparatus 1 may identify the target site using a luminance value of the ultrasonic image data IM1 or an index value (elastic modulus or viscosity) representing the tissue characterization obtained at each position (each pixel or pixel group) in the ultrasonic image data IM1.

The ultrasonic diagnostic apparatus 1 may identify the target site using at least two of the NLV, the luminance value of the ultrasonic image data IM1, and the index value representing the tissue characterization obtained at each position (each pixel or pixel group) in the ultrasonic image data IM1. That is, the ultrasonic diagnostic apparatus 1 may identify the target site by combining a plurality of pieces of information for identifying the target site, such as the NLV, the luminance value of the ultrasonic image data IM1, and the index value representing the tissue characterization.

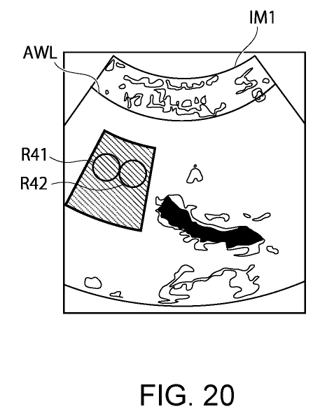

In the first to fourth embodiments, the first modification, and the second modification, the observation ROI and the measurement ROI are described using ATI (attenuation constant) as an example of the tissue characterization; however, this is not intended to be limiting. The observation ROI and the measurement ROI may be regions for observing the tissue characterization in the SWE, the SWD, or the like. FIG. 20 is a diagram illustrating an example of a case of setting an observation ROI and measurement ROIs in an SWE image. When the tissue characterization in the SWE is observed, the observation ROI is placed on ultrasonic image data of the subject such that an upper end of the observation ROI has a predetermined length (1.5 cm to 2.0 cm) from the deep portion of the abdominal wall. In addition, the ultrasonic image data of the subject in this case may be any of the B-mode image data representing a tissue shape, the Doppler image data representing blood flow dynamics (fluid dynamics), and the tissue characterization image data (ATI image data, SWE image data, SWD image data, NLV image data, or the like) representing the tissue characterization. The identification function 383 may obtain a target site (e.g., deep portion of the abdominal wall) from results of profiles of various information (e.g., tissue shape, fluid dynamics, or tissue characterization) on a plurality of scanning lines (transmission directions of ultrasonic waves (depth direction of the subject P)) included in the ultrasonic image data of the subject P. The example in which the calculation function 384 obtains the average value of the tissue characterization parameter of each position (each candidate region) as the index value representing the reliability of the tissue characterization parameter is described; however, this is not intended to be limiting. For example, the calculation function 384 may obtain the standard deviation, a variance, or the like (statistical value) of the tissue characterization parameter at each position (each candidate region), as the index value representing the reliability of the tissue characterization parameter. Specifically, the calculation function 384 may obtain the standard deviations representing the reliability of the tissue characterization represented by the SWE image data for a plurality of the candidate regions that are specified with the deep portion of the abdominal wall serving as a reference, and the setting function 385 may set, as the observation ROI, a candidate region having the smallest standard deviation among the results calculated by the calculation function 384. In the example illustrated in FIG. 20, a plurality of the measurement ROIs (R41, R42) having circular shapes are set into the observation ROI. When the plurality of measurement ROIs (R41, R42) are set, the statistical value of the tissue characterization parameter may be calculated for each measurement ROI (R41, R42), or one statistical value of the tissue characterization parameter based on the statistical values of the tissue characterization parameter calculated for the plurality of measurement ROIs(R41, R42) may be calculated.

In the first to fourth embodiments, the first modification, and the second modification described above, the identification function 383 identifies the deep portion of the abdominal wall (portion where the visceral peritoneum and the hepatic surface are located) as the target site; however, the target site is not limited to this. That is, the site identified as the target site by the identification function 383 is freely selected, and the identification function 383 may identify an anatomical feature site other than the deep portion of the abdominal wall.

In the first to fourth embodiments, the first modification, and the second modification described above, the identification function 383 obtains the target site from the results of the profiles of the tissue characterization parameter on the plurality of scanning lines; however, the identification function 383 can also obtain the target site from the results of the profiles of the B-mode data on the plurality of scanning lines.

In the first to fourth embodiments, the first modification, and the second modification described above, the width and length of each of the candidate regions are the same as the width and length of the observation ROI included in the ultrasonic image data; however, this is not intended to be limiting. In step S17 of the observation ROI setting processing, when setting the plurality of candidate regions to the ultrasonic image data, the calculation function 384 may adjust the width and length of each of the plurality of candidate regions and set the plurality of adjusted candidate regions to the ultrasonic image data.

In the first to fourth embodiments, the first modification, and the second modification described above, the case where the observation ROI and the measurement ROI are set to the two-dimensional ultrasonic image data is described; however, the embodiments and the modifications are not limited to this. That is, the first to fourth embodiments, the first modification, and the second modification can be applied also in a case of setting the observation ROI and the measurement ROI to three-dimensional ultrasonic image data.

Note that the word "processor" used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the storage 37. Note that programs may be configured to be directly integrated in the processor instead of being storing in the storage 37. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

According to at least one embodiment described above, the tissue characterization of the subject can be analyzed with high accuracy.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An ultrasonic diagnostic apparatus comprising processing circuitry configured to:
identify a target site included in ultrasonic image data of a subject;
calculate respective index values for a plurality of positions that are specified with the target site serving as a reference, the index values representing reliability of a tissue characterization parameter represented by the ultrasonic image data; and
set, based on the calculated results, a region of interest for observing the tissue characterization parameter onto an image represented by the ultrasonic image data.

2. The ultrasonic diagnostic apparatus of claim 1, wherein the processing circuitry is further configured to set, to the ultrasonic image data, a plurality of candidate regions for calculating the index values.

3. The ultrasonic diagnostic apparatus of claim 2, wherein the plurality of candidate regions are set to predetermined positions deeper than the target site.

4. The ultrasonic diagnostic apparatus of claim 2, wherein the index values representing the reliability of the tissue characterization parameter are statistical values indicating a variation of index values that represent the tissue characterization parameter in the candidate regions.

5. The ultrasonic diagnostic apparatus of claim 2, wherein the processing circuitry is further configured to:
set the plurality of candidate regions to the ultrasonic image data; and
calculate the respective index values for the plurality of positions by calculating the respective index values for the plurality of candidate regions.

6. The ultrasonic diagnostic apparatus of claim 1, wherein the target site is obtained from profiles of the tissue characterization parameter on a plurality of scanning lines or results of profiles of B-mode data on a plurality of scanning lines.

7. The ultrasonic diagnostic apparatus of claim 1, wherein the region of interest for observing the tissue characterization parameter is a measurement region of interest (ROI) in which statistical values of the tissue characterization parameter are measured.

8. The ultrasonic diagnostic apparatus of claim 1, wherein the region of interest for observing the tissue characterization parameter is an imaging ROI in which a color map based on the tissue characterization parameter is displayed.

9. The ultrasonic diagnostic apparatus of claim 1, wherein the tissue characterization parameter is an attenuation parameter that represents attenuation of an ultrasonic wave in a tissue.

10. The ultrasonic diagnostic apparatus of claim 1, wherein the tissue characterization parameter is an elasticity parameter that represents elasticity of a tissue.

11. The ultrasonic diagnostic apparatus of claim 1, wherein the tissue characterization parameter is a viscosity parameter that represents viscosity of a tissue.

12. The ultrasonic diagnostic apparatus of claim 1, wherein the target site is a deep portion of an abdominal wall.

13. The ultrasonic diagnostic apparatus of claim 1,
wherein the region of interest for observing the tissue characterization parameter is a measurement ROI in which a statistical value of the tissue characterization parameter is measured, and
wherein the processing circuitry is further configured to:
calculate the respective index values for the plurality of positions in an imaging ROI in which a color map based on the tissue characterization parameter is displayed; and
set the measurement ROI into the imaging ROI based on the calculated results.

14. The ultrasonic diagnostic apparatus of claim 7, wherein the processing circuitry is further configured to set the measurement ROI onto the image while the ultrasonic image data is being reproduced.

15. The ultrasonic diagnostic apparatus of claim 7, wherein the processing circuitry is further configured to set the measurement ROI onto the image while reproduction of the ultrasonic image data is stopped.

16. A medical image processing apparatus comprising
processing circuitry configured to:
identify a target site included in medical image data of a subject;
calculate respective index values for a plurality of positions that are specified with the target site serving as a reference, the index values representing reliability of a tissue characterization parameter represented by the medical image data; and
set, based on the calculated results, a region of interest for observing the tissue characterization parameter onto an image represented by the medical image data.

17. A non-transitory computer readable storage medium, which stores a program that causes a computer to execute processing comprising:
identifying a target site included in ultrasonic image data of a subject;
calculating respective index values for a plurality of positions that are specified with the target site serving as a reference, the index values representing reliability of a tissue characterization parameter represented by the ultrasonic image data; and
setting, based on the calculated results, a region of interest for observing the tissue characterization parameter onto an image represented by the ultrasonic image data.
